(19) United States
(12) Patent Application Publication
(10) Pub. No.: US 2018/0244927 A1
(43) Pub. Date: Aug. 30, 2018

(54) COATING COMPOSITION AND USES THEREOF

(71) Applicant: The Queen's University of Belfast, Belfast (GB)

(72) Inventors: Colin McCoy, Belfast (GB); Sean Gorman, Belfast (GB); David Jones, Belfast (GB); Daniel Corbett, Belfast (GB); Nicola Irwin, Belfast (GB)

(73) Assignee: The Queen's University of Belfast, Belfast (GB)

(21) Appl. No.: 15/556,255

(22) PCT Filed: Mar. 4, 2016
(86) PCT No.: PCT/EP2016/054679

§ 371 Date: Sep. 6, 2017

(30) Foreign Application Priority Data
Mar. 6, 2015 (GB) 1503820.1

(51) Int. Cl. C09D 4/06; C09D 5/00; A61L 29/04; A61L 29/08; A61L 29/14

(52) U.S. Cl. CPC C09D 4/06; C09D 5/00; A61L 29/041; A61L 2420/06; A61L 29/14; A61L 2400/10; A61L 2420/02; A61L 29/085

(57) ABSTRACT

The present invention provides a polymerizable coating composition comprising: (i) 1-vinyl-2-pyrrolidinone (NVP) and 2-hydroxyethylmethacrylate (2-HEMA) for forming a copolymer; (ii) a poly(vinyl pyrrolidone) (PVP) polymer having a molecular weight of at least 500,000 g·mol⁻¹; (iii) a co-polymerizable crosslinker for crosslinking the 1-vinyl-2-pyrrolidinone (NVP) and the 2-hydroxyethylmethacrylate (2-HEMA); (iv) a co-polymerizable initiator for initiating co-polymerisation of the 1-vinyl-2-pyrrolidinone (NVP) and 2-hydroxyethylmethacrylate (2-HEMA); (v) a surfactant selected from non-ionic triblock copolymers of ethylene oxide/propylene oxide, nonionic triblock copolymers of poly(propylene oxide) (PPO) and poly(ethylene oxide) (PEO), tetrablock copolymers of PPO and PEO; Triton X-100 and derivatives thereof, polyethylene glycols and derivatives thereof, PEO derivatives of sorbitan monolaurate, and combinations of any two or more thereof; (vi) a solvent, wherein a vinyl component is 1-vinyl-2-pyrrolidinone for forming the copolymer and the PVP polymer, and an acrylate component is 2-hydroxyethyl methacrylate for forming the copolymer; wherein the vinyl and acrylate components are present at a molar ratio of between 10:1 and 1:10; and wherein 20-80% (w/w) of the vinyl component is PVP polymer.

### FIELD OF THE INVENTION

This invention relates to a polymerizable coating composition comprising 1-vinyl-2-pyrrolidinone (NVP) and 2-hydroxyethyl methacrylate (2-HEMA) for forming a copolymer, poly(vinyl pyrrolidone) (PVP), a co-polymerizable crosslinker, a co-polymerizable initiator, a surfactant and a solvent, which is suitable for use in medical, and other, applications to reduce surface friction.

### BACKGROUND OF THE INVENTION

Intermittent self-catheterisation (ISC), involving the regular insertion of catheters into the bladder via the urethra by patients with poor control over their bladder function, for example those with incontinence or urinary retention problems, is increasingly preferred over the use of indwelling catheters to drain urine. This bladder management technique is associated with a lower risk of infection and offers patients a potentially greater degree of personal independence and self-care than the indwelling approach.

EP0382005 concerns the preparation of pigmented lacquers useful to form a colour effect in contact lenses. Claim 1 is directed to the preparation of pigmented lacquers or varnishes for colouring contact lenses. While the method does involve forming a copolymer from 1-vinyl-2-pyrrolidinone (NVP) and 2-hydroxyethyl methacrylate (2-HEMA) by photoinitiation, neither preformed poly(vinyl pyrrolidone) (PVP) polymer nor surfactant is present.

WO2009/023843 concerns polymers including an N-vinyl amide monomer and a dual functional monomer. Paragraph [0034]) lists many potential utilities for these polymers including medical device coatings. While the method of Claim 11 does involve forming a copolymer from at least 1-vinyl-2-pyrrolidinone (NVP) and 2-hydroxyethyl methacrylate (2-HEMA), neither poly(vinyl pyrrolidone) (PVP) polymer nor surfactant is present.

WO00/30698 concerns tissue/implant interactions. Page 20, lines 3-8 disclose a stent to keep blood vessels open following balloon angioplasty, the polymer layer containing microsphere-encapsulated drugs to prevent inflammation and restinosis. Examples 3-6 involve the determination, for HEMA-FOSA hydrogels prepared in Example 2, of hydrogel permeability; incorporation of growth factors; demonstration of neovascularization; and preparation of ion-exchange resin self-assemblies respectively. The hydrogels of Example 2 comprise HEMA, FOSA and AIBN, mixed with dioxane and then swollen in water/acetone mixtures. Neither preformed poly(vinyl pyrrolidone) (PVP) polymer nor surfactant is present.

US2004/209973 concerns silicone hydrogel contact lenses. Paragraph [0038] discloses a lens for daily wear that is coated with, amongst other components, HEMA. The lens coating composition also includes poly(acrylic acid), TRIS, DMA, mPDMS, NORBLOC, CGI, TEGDMA or D30 diluent.

U.S. Pat. No. 5,290,548 concerns surface modification of the plastic surface of an article. Claim 1 is directed to a method of surface modification by gamma irradiation as the method of polymerization. No preformed poly(vinyl pyrrolidone) (PVP) polymer or surfactant is present.

U.S. Pat. No. 4,842,597 concerns hydrophilic copolymers for wound dressings. No preformed poly(vinyl pyrrolidone) (PVP) polymer or surfactant is present in any of the Examples. Column 5, lines 56-67 discloses that its copolymers improve biocompatibility and reduce the formation of blood clots.

Regular insertion of poorly lubricated catheters, however, leads to a plethora of urethral complications, such as trauma, bleeding and inflammation, with consequential implications on patient morbidity. Accordingly, there is a need for strategies which increase lubricity of the device surface and consequentially decrease frictional forces upon device insertion and removal.

Solutions provided to date involve the application of lubricating agents, such as silicone or glycerin, to a device surface. These slippery coatings, however, render the device difficult for patients and/or physicians to manipulate. Furthermore, minimal interaction with the device substrate leads to ready loss of these lubricating agents from the surface, and their efficacy in minimising patient trauma, especially upon device removal, is therefore limited. Alternatively, the widespread adoption of hydrophilic PVP polymer coatings, the surfaces of which become slippery when wet, has transformed intermittent catheters from painful-to-use devices into products which are more lubricious and easier to insert.

In the hydrated state, the smooth and slippery coated surface of a catheter ensures lubrication of the urethra upon insertion. However, this effect is relatively short-lived. Coating materials rapidly dry out and are readily lost from the catheter surface leading to a loss of lubricity and reversion back to their pre-hydrated or uncoated state where catheterisation is a difficult, and painful, process. The current market standard PVP-coated catheters must be inserted and removed within approximately ten minutes, which is especially problematic considering the limited manual dexterity of many ISC users who are tetra- or paraplegic wheelchair drivers, or patients with e.g. late-stage multiple sclerosis or spina bifida. Despite their poor water retention and durability properties, these conventional coatings have not changed much in over a decade. Accordingly, lubricious materials capable of retaining water and adhering to the catheter substrate for longer periods of time are urgently needed to allow the patient sufficient time to insert and remove the catheter.

It would therefore be desirable to provide a lubricious, durable coating with an extended dry-out time in the form of a coating composition, ideally for application to ISC and other medical devices, to improve ease of device insertion and reduce trauma upon removal, and to reduce friction-related wear of mechanical components. Such a coating would also be suitable for use in non-medical environments, such as extending device lifespan by reducing friction-related wear of mechanical components, and various friction-reducing applications in, for example, agricultural settings.

### SUMMARY OF THE INVENTION

Accordingly, in a first aspect of the invention, there is provided a polymerizable coating composition comprising:

(i) 1-vinyl-2-pyrrolidinone (NVP) and 2-hydroxyethyl methacrylate (2-HEMA) for forming a copolymer;

(ii) a poly(vinyl pyrrolidone) (PVP) polymer having a molecular weight of at least 500,000 g·mol⁻¹

(iii) a co-polymerizable crosslinker for crosslinking the 1-vinyl-2-pyrrolidinone (NVP) and the 2-hydroxyethyl methacrylate (2-HEMA);

(iv) a co-polymerizable initiator for initiating co-polymerisation of the 1-vinyl-2-pyrrolidinone (NVP) and 2-hydroxyethyl methacrylate (2-HEMA);

(v) a surfactant selected from non-ionic triblock copolymers of ethylene oxide/propylene oxide, non-ionic triblock copolymers of poly(propylene oxide) (PPO) and poly(ethylene oxide) (PEO), tetrablock copolymers of PPO and PEO; Triton X-100 and derivatives thereof, polyethylene glycols and derivatives thereof, PEO derivatives of sorbitan monolaurate, and combinations of any two or more thereof;

(vi) a solvent;

wherein a vinyl component is 1-vinyl-2-pyrrolidinone for forming the copolymer and the PVP polymer, and an acrylate component is 2-hydroxyethyl methacrylate for forming the copolymer;

wherein the vinyl and acrylate components are present at a molar ratio of between 10:1 and 1:10; and

wherein 20-80% (w/w) of the vinyl component is PVP polymer.

No special precautions need to be taken to prevent copolymerization, other than to protect the composition from light if the co-polymerizable initiator is a photoinitiator or to maintain the composition at ambient temperature if the co-polymerizable initiator is a thermal initiator.

In a second aspect of the invention, there is provided a method of coating a surface with a polymerizable coating composition of the first aspect of the invention, the method comprising: contacting the composition of the first aspect of the invention with a surface; and irradiating or heating the composition on the surface. Without being bound by theory, it is expected that the coating composition of the first aspect of the invention should work on most metal, silicon dioxide- or carbon-containing surfaces and adheres well to glass, polyurethane, PVC and the like. Preferred surfaces include polyvinylchlorides (PVCs) and polyurethanes.

Optionally, the irradiating is with light at a wavelength of between 230 to 375 nm. Optionally, the heating is heating at between 60 to 90° C.

Optionally, the irradiating is with light at a wavelength of between 350 to 375 nm, between 230 to 250 nm, or between 270 to 290 nm. Further optionally, the irradiating is with light at a wavelength of about 365 nm.

Optionally, the surface comprises or consists of glass, polyurethane, and/or PVC. Optionally, the surface is a metal-containing-, silicon dioxide- or carbon-containing surface.

Optionally, during the irradiating or heating step, the coating composition covalently bonds to the surface during polymerisation of the coating composition.

Hereinafter refers to any aspect of the invention, including the above-mentioned first and second aspects of the invention.

Optionally, the vinyl and acrylate components are present at a molar ratio of between 5:1 and 1:5, optionally between 3:1 and 1:1, further optionally about 2:1.

Optionally, the vinyl component comprises between 25-70% (w/w) PVP polymer; optionally wherein the vinyl component comprises between 35-60% (w/w) PVP polymer; further optionally wherein the vinyl component comprises between 40-55% (w/w) PVP polymer; still further optionally wherein the vinyl component comprises about 50% (w/w) PVP polymer.

The PVP is soluble in water and other polar solvents. When dry, it is a light flaky hygroscopic powder, readily absorbing up to 40% of its weight in atmospheric water. PVP has a molecular mass in the range of 2,500-2,500,000 g·mol⁻¹. A PVP with a molecular mass of greater than 750,000 g·mol⁻¹ is preferred. A PVP with a molecular mass in the range of 1,000,000 and 1,500,000 g·mol⁻¹ is further preferred.

The inventors have found that the ratio of the NVP to the 2-HEMA in the copolymer, as defined herein, is important in dictating the resulting mechanical properties of the formed coatings. It has been found that the recited combination of NVP and 2-HEMA results in a copolymer coating possessing the required balance of flexibility and durability for application to devices such as catheters. Improved durability also means that the coating composition possesses superior adherent and cohesive properties and thus can resist the mechanical forces experienced by the device, e.g. during insertion and removal of a catheter. This represents a significant advantage over conventional lubricants which are readily “sloughed off” device surfaces, so that the devices therefore revert back to their uncoated states making them painful, and difficult, to insert and remove, arising from high frictional forces, therefore causing much patient trauma.

Optionally, the co-polymerizable crosslinker is present in an amount of about 0.1 to 5% (w/w); optionally 0.25 to 2.5% (w/w); further optionally about 1% (w/w); of the sum of components (i) to (v) of the composition. Further optionally, the co-polymerizable crosslinker is selected from ethyleneglycol-dimethacrylate, ethylene dimethacrylate, diethylene glycol diacrylate, diethylene diacrylate, bis[4-(2-hydroxy-3-methacryloyloxypropoxy)phenyl]sulfide, ethylidene-bis-3-(N-vinyl-2-pyrrolidone), methacryloxyethyl vinyl carbonate, ethylene glycol divinyl carbonate, allyl methacrylate, N,N′-methylenebisacrylamide, and 1,4-butanedioldiacrylate or a mixture thereof.

Optionally, the initiator is present in an amount of 0.1 to 10% (w/w); optionally 0.5 to 7.5% (w/w); further optionally 1 to 5% (w/w); still further optionally about 2% (w/w); of the sum of components (i) to (v) of the composition.

Further optionally, the initiator is selected from a photoinitiator and a thermal initiator.

Optionally, the photoinitiator is selected from one or more of a hydroxyketone photoinitiator, an amino ketone photoinitiator, a hydroxy ketone/benzophenone photoinitiator, a benzyldimethyl ketal photoinitiator, a phenylglyoxylate photoinitiator, an acyl phosphine oxide photoinitiator, an acyl phosphine oxide/alpha hydroxy ketone photoinitiator, a benzophenone photoinitiator, a phenylglyoxylate photoinitiator. Further optionally, the hydroxyketone photoinitiator is selected from 2,2-dimethoxy-1,2-diphenylethan-1-one, 2-hydroxy-2-methyl-1-phenyl-propan-1-one and 1-[4-(2-hydroxyethoxy)-phenyl]-2-hydroxy-2-methyl-1-propane-1-one. Still further optionally, the hydroxyketone photoinitiator is 1-[4-(2-hydroxyethoxy)-phenyl]-2-hydroxy-2-methyl-1-propane-1-one.

Optionally, the thermal initiator is selected from one or more of 2,2′-azobisisobutyronitrile (AIBN), benzoyl peroxide (BPO), ammonium persulfate/tetramethylethylenediamine (APS/TMEDA), and potassium persulfate/tetramethylethylenediamine (KPS/TEMDA).

Optionally, the PVP polymer has an average molecular weight (M_{w}) in the range of 500,000 to 1,500,000 g·mol⁻¹. Optionally, the average M_{w} of the PVP polymer is in the range of 1,000,000 to 1,500,000 g·mol⁻¹.

It has also been found that inclusion of PVP polymer, along with the copolymer comprising NVP and 2-HEMA, provides a composition having appropriate viscosity for coating of device surfaces, for example, by a dip-coating process.

Optionally, the surfactant is present in an amount of 0.1 to 20% (w/w); optionally 1 to 10% (w/w) or 1 to 15% (w/w); further optionally 2 to 10% (w/w) or 2.5 to 7.5% (w/w); still further optionally about 5% (w/w); of the sum of components (i) to (v) of the composition. Further optionally, the surfactant is selected from non-ionic triblock copolymers of poly(propylene oxide) (PPO) and poly(ethylene oxide) (PEO), tetrablock copolymers of PPO and PEO; Triton X-100 and derivatives thereof, polyethylene glycols and derivatives thereof, PEO derivatives of sorbitan monolaurate, and combinations of any two or more thereof. Still further optionally, the surfactant is a nonionic triblock copolymer composed of a central hydrophobic chain of poly(propylene oxide) flanked by two hydrophilic chains of poly(ethylene oxide). Optionally, the nonionic triblock copolymer comprises a poly(oxypropylene) molecular mass of 1,800 g·mol⁻¹ and a poly(oxyethylene) content of 80%.

Optionally, the surfactant is a poloxamer—a nonionic triblock copolymer composed of a central hydrophobic chain of polyoxypropylene (poly(propylene oxide)) flanked by two hydrophilic chains of polyoxyethylene(poly(ethylene oxide)). Poloxamers are also known by the trade names Synperonics, Pluronics, and Kolliphor and, for the Pluronic tradename, coding of these copolymers starts with a letter to define its physical form at room temperature (L=liquid, P=paste, F=flake (solid)) followed by two or three digits—the first digit (or two digits in a three-digit number) in the numerical designation, multiplied by 300, indicates the approximate molecular weight of the hydrophobe; and the last digit×10 gives the percentage polyoxyethylene content (e.g., L61 indicates a polyoxypropylene molecular mass of 1,800 g·mol⁻¹ and a 10% polyoxyethylene content).

By “components of the composition” is meant the components of the coating composition consisting of the vinyl component comprising NVP and PVP, 2-HEMA, the crosslinker, the initiator and the surfactant, and excluding the solvent.

Optionally, the surfactant is dispersed uniformly, or substantially uniformly, throughout the polymerizable coating composition.

Homogeneous or uniform dispersal of the surfactant in the polymerizable coating composition helps to ensure that the surfactant is sufficiently exposed to aqueous media when a coated device is “wetted” to achieve its lubricious effects. The water-binding capacity of the friction-reducing coating, from which the lubricious properties derive, is thought to be improved by the presence of surfactants in close proximity to the device surface, which close proximity is achieved by the uniform, or substantially uniform, distribution of the surfactant in the coating composition.

The recited surfactants are surface-active agents with hydrophilic groups which undergo hydrogen bonding with water molecules. Their presence in the coating composition increases the rate and extent of water uptake, and extends the time before which the coating dries out and becomes “tacky”. A “tacky” coating can cause discomfort and pain to a subject when a coated device, such as a catheter, is inserted or removed from the subject's body.

Optionally, the solvent is a mixture of water and at least one alcohol. Further optionally, the mixture of alcohol and water is in a ratio of between 10:1 and 1:10 (v/v), optionally between 5:1 and 1:5 (v/v), further optionally between 3:1 and 1:1 (v/v) or 1:1 to 5:1 (v/v) or 1:1 to 2:1 (v/v), still further optionally about 2:1 (v/v). Still further optionally, the alcohol is selected from propan-2-ol, methanol and ethanol, or a mixture thereof, optionally the alcohol is propan-2-ol.

Optionally, the final concentration of components (i) to (v) is between 25 to 50% (w/v of the coating composition); optionally 30 to 40% (w/v); further optionally about 37.3% (w/v).

The choice of an appropriate solvent is important in that it must be compatible with components (i) to (v) of the composition. For example, if an incompatible solvent is chosen, the surfactant will not homogeneously interpenetrate the coating composition and this will result in a poorly performing coating composition.

Optionally, the polymerizable coating composition further comprises one or more therapeutic drugs (such as, but not limited to, chlorhexidine) that, in use, may be eluted from the polymerizable coating composition.

In a third aspect of the invention, there is provided a method of coating a device, the method comprising:
(i) contacting a device to be coated with the coating composition of the first aspect of the invention;
(ii) drying the coating composition at an elevated temperature for 10 to 60 minutes; and
(ii) initiating polymerisation of the coating composition, optionally by irradiating the coated device with light at a wavelength of between 230 to 375 nm, or by heating the coated device at between 60 to 90° C.

As used herein, a device is any product having a surface and includes medical devices, as well as, apparatus for use in another field.

Optionally, contacting the device with the coating composition of the first aspect of the invention comprises dip-coating the device with the coating composition.

Optionally, the drying is at a temperature of between 40 and 70° C. for a period of between 10 and 60 minutes. Further optionally, the drying is at a temperature of 60° C. for a period of 20 minutes.

Optionally, the irradiating is with light at a wavelength of between 350 to 375 nm, between 230 to 250 nm, or between 270 to 290 nm. Further optionally, the irradiating is with light at a wavelength of about 365 nm.

In a fourth aspect of the invention, there is provided a device coated, wholly or in part, with the coating composition of the first aspect of the invention; or a device coated, wholly or in part, with the polymerized coating composition of the first aspect of the invention.

Optionally, in the method of the third aspect of the invention, or the device of the fourth aspect of the invention, the device is selected from urinary catheters, endotracheal tubes, coronary stents, angioplasty balloons, ureteral stents, contact lenses, vascular stents and cardiac pacemakers.

Embodiments of the present invention will now be described with the aid of the following examples.

### EXAMPLES

Materials

Coating Components and Suppliers: Poly(vinyl pyrrolidone) (PVP), specifically Kollidon 90F (K90F), and Pluronic F68 from BASF Chemical Corporation, Ludwigshafen, Germany; 1-vinyl-2-pyrrolidinone (NVP) and 2-hydroxyethyl methacrylate (2-HEMA) from Sigma-Aldrich, Gillingham, Dorset, UK. K90F is a PVP polymer having a molecular weight in the range of 1,000,000 to 1,500,000 g·mol⁻¹. Pluronic F68 is a non-ionic surfactant that is a difunctional block copolymer surfactant terminating in primary hydroxyl groups. Pluronic F68 has an average molecular weight of 8400 g·mol⁻¹. Propan-2-ol was obtained from Sigma-Aldrich, Gillingham, Dorset, UK. Photoinitiators initiate the photopolymerisation of chemically unsaturated prepolymers, e.g. unsaturated polyesters or acrylates, in combination with mono- or multifunctional monomers. The photoinitiators used were Irgacure 1173 from BASF Chemical Corporation, Ludwigshafen, Germany and Irgacure 2959 from Sigma-Aldrich, Gillingham, Dorset, UK. Irgacure 1173 is 2-hydroxy-2-methyl-1-phenyl-propan-1-one and Irgacure 2959 is 1-[4-(2-hydroxyethoxy)-phenyl]-2-hydroxy-2-methyl-1-propane-1-one.

Formulation of Acrylate-Based Copolymer Coating Compositions

Formulations 1-8 with a final concentration of coating components in solvent of 37.3% w/v, the compositions of which are described in Table 1, were prepared by mixing ethyleneglycol-dimethacrylate (EGDMA) crosslinker, 1-vinyl-2-pyrrolidinone (NVP), and 2-hydroxyethyl methacrylate (2-HEMA). The appropriate volumes of propan-2-ol and distilled water were added before the addition of Pluronic F68 and Kollidon 90F slowly with stirring. Finally, when all components were dissolved, Irgacure 1173 or Irgacure 2959 was added as the photoinitator.

PVC catheter substrates, previously etched in ethanol for 1 minute, were dipped in the coating formulation and irradiated with a 366 nm UV lamp (400 W) for three to five minutes to copolymerise the NVP and 2-HEMA.

The following components were kept constant in the tested formulations:

1% w/w EGDMA

2% w/w Irgacure 1173 or 2% w/w Irgacure 2959

5% w/w Pluronic F68

Solvent: 2:1 mix of propan-2-ol/water (v/v)

Final concentration: 37.3% w/v (weight of the coating components; the balance of 62.7% (w/v) being solvent).

A concentration of 5% w/w Pluronic was preferred in haptic assessments over the 10% w/w Pluronic-containing coatings. A volume ratio (2:1) was selected based on haptic assessments of catheter coatings formulated with a range of solvent ratios from 100% water to 100% propan-2-ol.

TABLE 1
Composition of Coating Formulations 1-8

| | Mass of Coating Components (g) | | | | | | Volume of | |
|---|---|---|---|---|---|---|---|---|
| | | Irgacure | | | | Kollidon | Solvent (mL) | |
| | | 1173 or | | | 2- | 90F | Propan- | |
| | EGDMA | Irgacure | Pluronic | NVP | HEMA | (PVP) | 2-ol | Water |
| Formulation | (g) | 2959 (g) | F68 (g) | (g) | (g) | (g) | (mL) | (mL) |
| 1 | 0.1 | 0.2 | 0.5 | 2.9 | 3.4 | 2.9 | 17.9 | 8.9 |
| 2 | 0.1 | 0.2 | 0.5 | 5.8 | 3.4 | — | 17.9 | 8.9 |
| 3 | 0.1 | 0.2 | 0.5 | 4.35 | 3.4 | 1.45 | 17.9 | 8.9 |
| 4 | 0.1 | 0.2 | 0.5 | 1.45 | 3.4 | 4.35 | 17.9 | 8.9 |
| 5 | 0.1 | 0.2 | 0.5 | — | 3.4 | 5.8 | 17.9 | 8.9 |
| 6 | 0.1 | 0.2 | 0.5 | 2.14 | 4.96 | 2.14 | 17.9 | 8.9 |
| 7 | 0.1 | 0.2 | 0.5 | 4.12 | 0.96 | 4.12 | 17.9 | 8.9 |
| 8 | 0.1 | 0.2 | 0.5 | 0.36 | 8.48 | 0.36 | 17.9 | 8.9 |

Changing the photoinitiator from Irgacure 1173 to Irgacure 2959 had no effect on the coating properties or data described in the cited examples. Irgacure 2959 is preferred in terms of compatibility and reliability.

Methods

Coating formulations were characterised by haptic assessment, friction testing and dry-out studies.

Haptic assessments involved hydrating the coated catheter segments in dH₂O for 30 seconds, after which the surface was rubbed between fingers. A rating was given for each coating on a slipperiness scale where 0, 1 or 2 represented not slippery, slippery, and very slippery, respectively, and on a thickness scale where 0, 1 or 2 represented thin, ideal coating thickness, and thick, respectively.

Friction testing was performed according to ASTM test method D-1894 with a ChemInstruments COF-1000 Coefficient of Friction Tester. This involved fixing PVC samples coated with the respective formulations by the dip-coating procedure to the underside of a test sled (219 g), then hydrating in dH₂O for 30 seconds. Excess water was wiped off and the sled was placed on the test platform and attached to the load cell mount. The force required to pull the sled at a speed of 15 cm/min over the test platform was measured. The static frictional force (Fₛ) was observed as a peak at the beginning of motion in the graph displaying frictional force versus distance travelled, and the static coefficient of friction (μₛ) between each surface pressed together by a normal force (N) was calculated from the Amonton/Coulomb friction law:

μₛ=Fₛ/N

As motion of the sled over the platform continued, the dynamic force of friction (F_{d}) acted in parallel to the surface to oppose the net applied force. The dynamic coefficient of friction (μ_{d}) was calculated as follows:

μ_{d}=F_{d}/N

where N, again, represents the normal force acting perpendicular to the surface.

Dry-out studies were performed with PVC catheter segments coated by the dip-coating procedure. Samples were immersed in dH₂O for 30 seconds, weighed and left to dry at ambient temperature. At designated intervals, the coated PVC samples were reweighed until there was no further change in mass. The weight percentage of water within the hydrated coating was calculated at each time interval according to the following equation:

Water Content (%)=(Mₜ−M₀)/Mₜ×100

where Mₜ and M₀ represent the sample mass at time t and the dried sample mass respectively.

Results

Formulations 2, 3, 1, 4 and 5 all comprise the same concentration of 2-HEMA but were compared to see the effect of an increasing proportion of PVP on the slipperiness, thickness, friction and dry-out of the coating formulations, as demonstrated in Tables 2, 3 and 4 below. Formulations 2, 3, 1, 4 and ultimately demonstrate that replacing 50% w/w of NVP with Kollidon 90F (PVP) was optimal in terms of coating lubricity and thickness.

TABLE 2
The Effect of Changing the Proportion of NVP Replaced by PVP on
Haptic Assessment Scores for Coating Slipperiness and Thickness

| % w/w of | | | |
|---|---|---|---|
| NVP replaced | | | |
| by PVP | Formulation | Slipperiness | Thickness |
| 0 | 2 | 0 | 0 |
| 25 | 3 | 1 | 0 |
| 50 | 1 | 2 | 1 |
| 75 | 4 | 1 | 2 |
| 100 | 5 | 1 | 2 |

As the proportion of NVP replaced by PVP increased, an increase in coating thickness was observed. Replacing 50% w/w of NVP with PVP was demonstrated to be optimal for coating slipperiness.

Formulation 5 (100% PVP; 0% NVP) was found to be too thick to be used as a catheter coating so this was not taken forward for further characterisation of friction and dry-out properties. Lower concentrations of PVP (Kollidon 90F) were tested in formulations 4, 1 and 3. However, the other formulations may nevertheless be used to coat other types of surfaces and devices in respect of which the slipperiness and thickness of the respective formulations are appropriate.

TABLE 3
The Effect of Changing the Proportion of NVP Replaced
by PVP in the Coating Formulations on Static and
Dynamic Coefficient of Friction Values

| % w/w of | | Static coefficient | Kinetic coefficient |
|---|---|---|---|
| NVP replaced | | of friction values | of friction values |
| by PVP | Formulation | (Mean ± SD) | (Mean ± SD) |
| 0 | 2 | 0.185 ± 0.060 | 0.237 ± 0.019 |
| 25 | 3 | 0.142 ± 0.021 | 0.159 ± 0.020 |
| 50 | 1 | 0.051 ± 0.017 | 0.087 ± 0.021 |
| 75 | 4 | 0.112 ± 0.047 | 0.142 ± 0.064 |
| 100 | 5 | — | — |

The lowest values for the static and dynamic coefficient of friction were obtained by formulation 1 with 50% w/w of NVP replaced by PVP, and the highest friction values were obtained for formulation 2 (comparative formulation) which contained no preformed PVP. Formulations 3, 4, 6 and 7 are also useful as catheter coatings—they are similar to formulation 1, but the static and kinetic coefficient of friction values are slightly higher.

TABLE 4
The Effect of Changing the Proportion of NVP
Replaced by PVP on the Retained Water Content
of the Coated Catheters after 10 minutes

| % w/w of | | Retained water content |
|---|---|---|
| NVP replaced | | after 10 min (% of total |
| by PVP | Formulation | taken up) (Mean ± SD) |
| 0 | 2 | 49.2 ± 11.0 |
| 25 | 3 | 52.1 ± 12.8 |
| 50 | 1 | 76.1 ± 6.5 |
| 75 | 4 | 90.4 ± 2.1 |
| 100 | 5 | — |

After a dry-out period of 10 min, formulations 1 and 4 retained more than 75% of the water initially taken up in the 30 s hydration period. The dry-out period of formulation 4 was longer than formulation 1, however the thickness of the former formulation when coated on PVC catheters makes it less suitable as a candidate coating for intermittent catheters.

With a constant proportion of NVP replaced by PVP of 50% w/w, the effect of changing the molar ratio of NVP to 2-HEMA on the slipperiness, thickness, friction and dry-out of the coating formulations is demonstrated in Tables 5, 6 and 7.

TABLE 5
The Effect of Changing the Molar Ratio of NVP to 2-
HEMA in the Coating Formulations on Haptic Assessment
Scores of Coating Slipperiness and Thickness

| Molar ratio of | | | |
|---|---|---|---|
| NVP:2-HEMA | Formulation | Slipperiness | Thickness |
| 1:10 | 8 | 0 | 0 |
| 1:1 | 6 | 1 | 0 |
| 2:1 | 1 | 2 | 1 |
| 10:1 | 7 | 1 | 2 |

The optimal coating slipperiness was found with a molar ratio of NVP to 2-HEMA of 2:1. As the molar ratio of NVP increased, the coating thickness increased as a result of the higher content of preformed PVP in the formulations.

TABLE 6
The Effect of Changing the Molar Ratio of NVP
to 2-HEMA in the Coating Formulations on Static
and Dynamic Coefficient of Friction Values

| | | Static coefficient | Kinetic coefficient |
|---|---|---|---|
| Molar ratio of | | of friction values | of friction values |
| NVP:2-HEMA | Formulation | (Mean ± SD) | (Mean ± SD) |
| 1:10 | 8 | 0.249 ± 0.081 | 0.204 ± 0.051 |
| 1:1 | 6 | 0.099 ± 0.018 | 0.124 ± 0.035 |
| 2:1 | 1 | 0.051 ± 0.017 | 0.087 ± 0.021 |
| 10:1 | 7 | 0.116 ± 0.020 | 0.209 ± 0.044 |

The lowest values for the static and dynamic coefficient of friction were obtained by formulation 1 with a 2:1 molar ratio of NVP to 2-HEMA.

TABLE 7
The Effect of Changing the Molar Ratio of NVP to 2-
HEMA in the Coating Formulations on the Retained Water
Content of the Coated Catheters after 10 minutes

| | | Retained water content at |
|---|---|---|
| Molar ratio of | | 10 min (% of total taken |
| NVP:2-HEMA | Formulation | up) (Mean ± SD) |
| 1:10 | 8 | 40.1 ± 12.7 |
| 1:1 | 6 | 77.3 ± 3.0 |
| 2:1 | 1 | 76.1 ± 6.5 |
| 10:1 | 7 | 85.7 ± 0.9 |

After a dry-out period of 10 min, formulations 1, 6 and 7 retained more than 75% of the water initially taken up in the 30 s hydration period. Despite their longer dry-out periods, formulations 6 and 7 were considered less suitable as candidate coatings for intermittent catheters because of their thickness when coated on PVC catheters.

Formulations 3, 4, 6 and 7 are also useful as catheter coatings—they are similar to formulation 1, but the static and kinetic coefficient of friction values are slightly higher.

### CONCLUSIONS

The haptic assessment, friction and dry-out data obtained for the tested formulations supports the requirement for the presence of the copolymer (NVP: 2-HEMA in a molar ratio of between 10:1 and 1:10) and the PVP Kollidon 90F polymer (replacing 20-80% of the NVP component) to produce a lubricious, durable coating with an extended dry-out time.

1. A polymerizable coating composition comprising:
(i) 1-vinyl-2-pyrrolidinone (NVP) and 2-hydroxyethyl methacrylate (2-HEMA) for forming a copolymer;
(ii) a poly(vinyl pyrrolidone) (PVP) polymer having a molecular weight of at least 500,000 g·mol⁻¹;
(iii) a co-polymerizable crosslinker for crosslinking the 1-vinyl-2-pyrrolidinone (NVP) and the 2-hydroxyethyl methacrylate (2-HEMA);
(iv) a co-polymerizable initiator for initiating co-polymerisation of the 1-vinyl-2-pyrrolidinone (NVP) and 2-hydroxyethyl methacrylate (2-HEMA);
(v) a surfactant selected from non-ionic triblock copolymers of ethylene oxide/propylene oxide, non-ionic triblock copolymers of poly(propylene oxide) (PPO) and poly(ethylene oxide) (PEO), tetrablock copolymers of PPO and PEO; Triton X-100 and derivatives thereof, polyethylene glycols and derivatives thereof, PEO derivatives of sorbitan monolaurate, and combinations of any two or more thereof;
(vi) a solvent,
wherein a vinyl component is 1-vinyl-2-pyrrolidinone for forming the copolymer and the PVP polymer, and an acrylate component is 2-hydroxyethyl methacrylate for forming the copolymer;
wherein the vinyl and acrylate components are present at a molar ratio of between 10:1 and 1:10; and
wherein 20-80% (w/w) of the vinyl component is PVP polymer.

2. The polymerizable coating composition of claim 1, wherein the vinyl and acrylate components are present at a molar ratio of between 5:1 and 1:5, optionally between 3:1 and 1:1, further optionally about 2:1.

3. The polymerizable coating composition of claim 1 or 2, wherein the vinyl component comprises between 25-70% (w/w) PVP polymer;
optionally wherein the vinyl component comprises between 35-60% (w/w) PVP polymer;
further optionally wherein the vinyl component comprises between 45-55% (w/w) PVP polymer;
still further optionally wherein the vinyl component comprises about 50% (w/w) PVP polymer.

4. The polymerizable coating composition of any one of the preceding claims, wherein the co-polymerizable crosslinker is present in an amount of about 0.1 to 5% (w/w); optionally 0.25 to 2.5% (w/w); further optionally about 1% (w/w), of components (i) to (v) of the composition.

5. The polymerizable coating composition of claim 4, wherein the co-polymerizable crosslinker is selected from ethyleneglycol-dimethacrylate, ethylene dimethacrylate, diethylene glycol diacrylate, diethylene diacrylate, bis[4-(2-hydroxy-3-methacryloyloxypropoxy)phenyl]sulfide, ethylidene-bis-3-(N-vinyl-2-pyrrolidone), methacryloxyethyl vinyl carbonate, ethylene glycol divinyl carbonate, allyl methacrylate, N,N′-methylenebisacrylamide, and 1,4-butanedioldiacrylate or a mixture thereof.

6. The polymerizable coating composition of any one of the preceding claims, wherein the co-polymerizable initiator is present in an amount of 0.1 to 10% (w/w); optionally 0.5 to 7.5% (w/w); further optionally 1 to 5% (w/w); still further optionally about 2% (w/w), of components (i) to (v) of the composition.

7. The polymerizable coating composition of any one of the preceding claims, wherein the co-polymerizable initiator is selected from a photoinitiator and a thermal initiator.

8. The polymerizable coating composition of claim 7, wherein the photoinitiator is selected from one or more of a hydroxyketone photoinitiator, an amino ketone photoinitiator, a hydroxy ketone/benzophenone photoinitiator, a benzyldimethyl ketal photoinitiator, a phenylglyoxylate photoinitiator, an acyl phosphine oxide photoinitiator, an acyl phosphine oxide/alpha hydroxy ketone photoinitiator, a benzophenone photoinitiator, a phenylglyoxylate photoinitiator.

9. The polymerizable coating composition of claim 8, wherein the hydroxyketone photoinitiator is selected from 2,2-dimethoxy-1,2-diphenylethan-1-one, 2-hydroxy-2-methyl-1-phenyl-propan-1-one and 1-[4-(2-hydroxyethoxy)-phenyl]-2-hydroxy-2-methyl-1-propane-1-one.

10. The polymerizable coating composition of claim 7, wherein the thermal initiator is selected from one or more of 2,2′-azobisisobutyronitrile (AIBN), benzoyl peroxide (BPO), ammonium persulfate/tetramethylethylenediamine (APS/TMEDA), and potassium persulfate/tetramethylethylenediamine (KPS/TEMDA).

11. The polymerizable coating composition of any one of claims 1 to 10, wherein the PVP polymer has an average molecular weight (M_{w}) in the range of 1,000,000 to 1,500,000 g·mol⁻¹.

12. The polymerizable coating composition of any one of claims 1 to 11, wherein the surfactant is present in an amount of 0.1 to 20% (w/w); optionally 1 to 15% (w/w); further optionally 2 to 10% (w/w); still further optionally about 5% (w/w), of components (i) to (v) of the composition.

13. The polymerizable coating composition of any one of claims 1 to 12, wherein the surfactant is selected from non-ionic triblock copolymers of poly(propylene oxide) (PPO) and poly(ethylene oxide) (PEO), tetrablock copolymers of PPO and PEO; Triton X-100 and derivatives thereof, polyethylene glycols and derivatives thereof, PEO derivatives of sorbitan monolaurate, and combinations of any two or more thereof.

14. The polymerizable coating composition of any one of claims 1 to 13, wherein the surfactant is a nonionic triblock copolymer composed of a central hydrophobic chain of poly(propylene oxide) flanked by two hydrophilic chains of poly(ethylene oxide).

15. The polymerizable coating composition of claim 14, wherein the nonionic triblock copolymer comprises a poly(oxypropylene) molecular mass of 1,800 g·mol⁻¹ and a poly(oxyethylene) content of 80%.

16. The polymerizable coating composition of any one of claims 1 to 15, wherein the solvent is a mixture of water and at least one alcohol.

17. The polymerizable coating composition of claim 16, wherein the mixture of alcohol and water within the solvent is in a ratio of between 10:1 and 1:10 (v/v), optionally between 5:1 and 1:1 (v/v), further optionally between 3:1 and 1:1 (v/v), still further optionally about 2:1 (v/v) of the solvent.

18. The polymerizable coating composition of claim 16 or 17, wherein the alcohol is selected from propan-2-ol, methanol and ethanol, or a mixture thereof, optionally the alcohol is propan-2-ol.

19. The polymerizable coating composition of any one of claims 1 to 18, wherein the final concentration of components (i) to (v) is between 25 to 50% (w/v) of the coating composition; optionally 30 to 40% (w/v); further optionally about 37.3% (w/v).

20. A method of coating a surface with a polymerizable coating composition of any one of claims 1 to 19, the method comprising:
contacting the composition of any one of claims 1 to 19 with a surface;
drying the composition at an elevated temperature optionally at a temperature of between 40 and 70° C., for 10 to 60 minutes; and
irradiating or heating the composition on the surface, optionally by irradiating the coated surface with light at a wavelength of between 230 to 375 nm, or by heating the coated surface at between 60 to 90° C.

21. The method of claim 20, wherein the NVP and the 2-HEMA are present at a molar ratio of between 5:1 and 1:5; further optionally between 3:1 and 1:1; still further optionally about 2:1.

22. A method of coating a device, the method comprising:
(i) contacting a device to be coated with the polymerizable coating composition of any one of claims 1 to 19;
(ii) drying the coating composition, optionally at a temperature of between 40 and 70° C. for a period of 10 to 60 minutes; and
(ii) initiating polymerization of the coating composition, optionally by irradiating the coated device with light at a wavelength of between 230 to 375 nm, or heating the coated device at between 60 to 90° C.

23. The method of claim 22, wherein contacting the device with the polymerizable coating composition of any one of claims 1 to 19 comprises dip-coating the device with the polymerizable coating composition.

24. The method of claim 20 or 23, wherein the irradiating is with light at a wavelength of between 350 to 375 nm, between 230 to 250 nm, or between 270 to 290 nm.

25. The method of any one of claims 20 to 24, wherein the irradiating is with light at a wavelength of about 365 nm.

26. The method of any one of claims 20 to 25, wherein the drying is at a temperature of between 40 and 70° C. for a period of 10 to 60 minutes.

27. A device coated, wholly or in part, with the polymerizable coating composition of any one of claims 1 to 19.

28. A device coated, wholly or in part, with the polymerized coating composition of any one of claims 1 to 19.

29. The method of any one of claims 22 to 26, or the device of claim 27 or 28, wherein the device is selected from urinary catheters, endotracheal tubes, coronary stents, angioplasty balloons, ureteral stents, contact lenses, vascular stents and cardiac pacemakers.
